(19) United States
(12) Patent Application Publication
(10) Pub. No.: US 2014/0358188 A1
(43) Pub. Date: Dec. 4, 2014

(54) ORTHOPEDIC TAMP AND BONE STABILIZATION MATERIAL DELIVERY DEVICE

(71) Applicant: Mind Rocket, Inc., Colorado Springs, CO (US)

(72) Inventors: Michael C. Larson, Colorado Springs, CO (US); Jesse R. McClure, Colorado Springs, CO (US); Richard M. Meinig, Colorado Springs, CO (US); Joe Griebel, Colorado Springs, CO (US)

(73) Assignee: Mind Rocket, Inc., Colorado Springs, CO (US)

(21) Appl. No.: 14/289,516

(22) Filed: May 28, 2014

Related U.S. Application Data
(60) Provisional application No. 61/827,997, filed on May 28, 2013.

(51) Int. Cl. A61B 17/92

(52) U.S. Cl. CPC A61B 17/92; A61B 2017/924
USPC 606/86 R

(57) ABSTRACT

An orthopedic bone tamp and bone stabilization material delivery device is operable to reduce depressed bone fragments to an elevated position and concurrently introduce to the fracture zone bone stabilization material so as to stabilize the newly positioned fragment. A bone tamp comprising a cannula having a tubular wall with outside diameter and inside diameters and defining a distal and proximal ends includes a bone displacement face associated with the closed distal end. A lumen defined by the walls of the cannula extends from the proximal end and terminates at the distal end face where it is associated with several apertures. The displacement face of the cannula is configured to operably manipulate and reduce bone fragments to an elevated position while the apertures and lumen can concurrently deliver to the fracture zone a bone stabilization material.

### RELATED APPLICATION

The present application relates to and claims the benefit of priority to U.S. Provisional Patent Application No. 61/827,997 filed May 28, 2013 which is hereby incorporated by reference in its entirety for all purposes as if fully set forth herein.

### BACKGROUND OF THE INVENTION

1. Field of the Invention

Embodiments of the present invention relate, in general, to orthopedic surgical devices and methodology and more particularly to a bone tamp operable to position fractured bones while concurrently introducing bone stabilization material.

2. Relevant Background

A bone fracture is a condition in which there is a break in the continuity of a bone. A fracture may be the result of a high force impact or longer period of enduring stress. In certain conditions an underlying medical condition can weaken the bone so that a trivial injury can cause a severe break.

The natural process of healing a fracture starts when the injured bone and surrounding tissues bleed, forming a fracture hematoma. The blood coagulates to form a blood clot situated between the broken fragments. Within a few days blood vessels grow into the jelly-like matrix of the blood clot. The new blood vessels bring phagocytes to the area that gradually removes the non-viable material. The blood vessels also bring fibroblasts in the walls of the vessels and these multiply and produce collagen fibers. In this way the blood clot is replaced by a matrix of collagen. Collagen's rubbery consistency allows bone fragments to move only a small amount unless severe or persistent force is applied.

At this stage, some of the fibroblasts begin to lay down bone matrix in the form of collagen monomers. These monomers spontaneously assemble to form the bone matrix, for which bone crystals (calcium hydroxyapatite) are deposited in, amongst, and in the form of insoluble crystals. This mineralization of the collagen matrix stiffens it and transforms it into bone. Indeed, bone is a mineralized collagen matrix; if the mineral is dissolved out of bone, it becomes rubbery. Healing bone callus is on average sufficiently mineralized to show up on an X-ray within 6 weeks in adults and less in children. This initial “woven” bone does not have the strong mechanical properties of mature bone. A process of remodeling replaces the woven bone by mature “lamellar” bone. The whole process can take up to 18 months, but in adults the strength of the healing bone is usually 80% of normal by 3 months after the injury.

There are many types of fractures, but the main categories are displaced, non-displaced, open, and closed. Displaced and non-displaced fractures refer to the way the bone breaks.

In a displaced fracture, the bone snaps into two or more parts and moves so that the two ends or pieces are not lined up. If the bone is in many pieces, it is called a comminuted fracture. In a non-displaced fracture, the bone cracks either part or all of the way through, but does not move and maintains its proper alignment.

Fracture reduction is the process by which the bones are realigned for healing. In close reduction the bone is “set” without surgery or without invasive treatment to the bone. In open reduction the bone fragments are exposed surgically and thereafter aligned.

A bone tamp is a device used in orthopedic surgery to reduce fractures and manage bone grafts. This type if instrument can be used inside a bone to elevate depressed areas after a fracture, assisting with the stabilization process. When there are depressions at the surface of a fractured or damaged bone around the sites of joints in the body, a bone tamp can be used to reposition the fracture pieces. If not positioned properly these fractured pieces can increase the risk of later developing arthritis, in addition to being painful for the patient. The surgeon thus uses the bone tamp to restore the shape of the bone, reducing the fracture so it can heal.

Bones are not uniformly solid material. Bones include a hard outer layer composed of compact bone tissue. This dense portion of the bone gives bone their smooth white solid appearance. The interior of the bone is a trabecular bone tissue. Such tissue is an open porous network of cancellous or spongy bone. While composing only 20% of the weight, this portion of the bone possesses nearly 10 times the surface area of compact bone.

During a depression fracture of a bone surface such as at a joint, the fractured pieces of the compact bone can depress into the softer trabecular tissue. The trabecular tissue is compressed such that upon elevation of the fractured compact pieces to their normal, pre-fractured positions, left behind is a void. Currently a tamp is used to position the fractured bone components to their natural state followed by an injection into the fracture zone of material to stabilize the area so that the fractured components can heal. Unfortunately during removal of the tamp the fractured pieces tend to return to their fractured position and upon injection of the stabilizing material, constrained to heal in an improper position.

A need therefore exists for a device, methodology and surgical kit, to assist medical professionals in the placement of fractured bone components into their natural, stabilized position for healing while concurrently addressing the void in trabecular bone tissue. What is needed is a combined orthopedic bone tamp and bone stabilizer delivery device that can reposition the fragments of fractured bones and concurrently fill the cavities within the trabecular tissue to provide a stable healing environment wherein the compact bone pieces are closely aligned to their natural “pre-fractured” position. These and other deficiencies of the prior art are addressed by one or more embodiments of the present invention.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### SUMMARY OF THE INVENTION

An orthopedic bone tamp and bone stabilization material delivery device of the present invention is operable to reduce depressed bone fragments to an elevated position while concurrently introduce to the fracture zone bone stabilization material so as to stabilize the newly positioned fragment.

According to one embodiment of the present invention a bone tamp comprising a cannula having a tubular wall with outside diameter and inside diameters and defining a distal and proximal ends includes a bone displacement face associated with the closed distal end. A lumen defined by the walls of the cannula extends from the proximal end and terminates at the distal end face where it is associated with several apertures. The displacement face of the cannula is configured to operably manipulate and reduce bone fragments to an elevated position while the apertures and lumen can concurrently deliver to the fracture zone a bone stabilization material.

The bone tamp and bone stabilization material delivery device of the present invention can further include a variety of bone displacement faces including planar, concave, convex and the like. The cannula is configured to interchangeably couple to a plurality of accessories, such as a handle by which to manipulate the cannula within the fracture zone, as well as a source for bone stabilization material. Significantly the exchange of the accessories associated with the cannula can occur without removal or repositioning of the cannula.

According to another embodiment of the present invention, a delivery tube, having an external diameter less than the interior diameter of the lumen within the cannula, can be inserted into the cannula to facilitate the delivery of the bone stabilization material. Moreover the delivery tube can include an internal helix or similar perturbations operable to support mixing of a multi-component bone stabilization material. Upon introduction of the various components of the bone stabilization material to the delivery tube, the components are mixed as they traverse the cannula so as to arrive at the apertures near the distal end fully activated.

A method for repositioning fracture bone components using an orthopedic bone tamp and bone stabilization material delivery device, according to the present invention, includes establishing an opening in a bone through which the bone tamp can be used to reposition fractured bone components to their natural position. The cannula is thereafter coupled to a source of bone stabilization material. This material is then delivered through the lumen and apertures of the cannula to the fracture zone to stabilize the newly reduced bone fragment. Significantly, the delivery of the bone stabilization material can occur without removal or repositioning of the cannula enabling the displacement face of the cannula to support the elevated bone fragment until introduction of the bone stabilization material.

According to another embodiment of the present invention a bone reconstruction kit includes, among other things, a bone tamp and bone stabilization delivery device. The kit includes a cannula defining a lumen traversing the proximal end of the cannula and termination short of the distal end. The distal end of the cannula includes a bone displacement face operable to manipulate bone fragments from a depressed to an elevated or reduced position. The kit further includes a source of bone stabilization material and a bone stabilization delivery system operable to delivery bone stabilization material from the source to the proximal end of the cannula. The kit further includes a handle or similar assessor operable to manipulate the bone tamp and bone displacement face so as to reposition bone fragments to a new normal position. A coupling system proximate to the proximal end of the cannula is also included in the kit that is operable to couple the cannula to a plurality of devices including the source of bone stabilization material and manipulation handle. These and other features of the present invention are described in detail by way of example below.

The features and advantages described in this disclosure and in the following detailed description are not all-inclusive. Many additional features and advantages will be apparent to one of ordinary skill in the relevant art in view of the drawings, specification, and claims hereof. Moreover, it should be noted that the language used in the specification has been principally selected for readability and instructional purposes and may not have been selected to delineate or circumscribe the inventive subject matter; reference to the claims is necessary to determine such inventive subject matter.

### BRIEF DESCRIPTION OF THE DRAWINGS

The aforementioned and other features and objects of the present invention and the manner of attaining them will become more apparent, and the invention itself will be best understood, by reference to the following description of one or more embodiments taken in conjunction with the accompanying drawings, wherein:

FIG. 1 shows a perspective image of an orthopedic tamp and bone stabilization material delivery device according to one embodiment of the present invention;

FIG. 2 shows a top-cutaway view of one embodiment of an orthopedic tamp and bone stabilization material delivery device according to the present invention;

FIG. 3 is a right perspective view of one embodiment of another embodiment of an orthopedic tamp and bone stabilization material delivery device according to the present invention;

FIG. 4A shows an expanded cutaway view of a distal end of an orthopedic tamp and bone stabilization material delivery device with a planar bone displacement face according to one embodiment of the present invention;

FIG. 4B shows an expanded cutaway view of a distal end of an orthopedic tamp and bone stabilization material delivery device with a concave bone displacement face according to one embodiment of the present invention;

FIG. 4C shows an expanded cutaway view of a distal end of an orthopedic tamp and bone stabilization material delivery device with a convex bone displacement face according to one embodiment of the present invention;

FIG. 5 shows one embodiment of an orthopedic bone tamp and bone stabilization material delivery device of the present invention combined with a bone manipulation handle and stabilization material delivery apparatus;

FIG. 6 is an expanded view of the coupling system between the orthopedic bone tamp and bone stabilization material delivery device and the handle according to one embodiment of the present invention;

FIG. 7 is a side view of an orthopedic bone tamp and bone stabilization material delivery device combined with a bone stabilization material source and mixing helix according to one embodiment of the present invention;

FIG. 8 is a top view of a bone stabilization material mixing helix according to one embodiment of the present invention;

FIG. 9 is an expanded cut away top and end view of a bone stabilization material mixing helix according to one embodiment of the present invention;

FIG. 10 is a detailed cut away view of the distal end of the orthopedic bone tamp and bone stabilization material delivery device combined with the bone stabilization material mixing helix according to one embodiment of the present invention;

FIG. 11 is a side cut away view of a bone stabilization material source syringe according to one embodiment of the present invention;

FIG. 12 shows insertion of an orthopedic bone tamp and bone stabilization material delivery device of the present invention inserted into a tibia for treatment of a depression fracture of the tibial plateau;

FIG. 13 shows the fractured bone pieces of a depression fracture of the tibial plateau repositioned using a orthopedic bone tamp and bone stabilization material delivery device according to one embodiment of the present invention;

FIG. 14 shows the repositioned fractured bone pieces of a depression fracture of a tibial plateau supported by bone stabilization material delivered using an orthopedic bone tamp and bone stabilization material delivery device of the present invention;

FIG. 15 shows a depression fracture of the tibial plateau reduced by one embodiment of an orthopedic bone tamp and bone stabilization material delivery device of the present invention;

FIG. 16 is a flowchart of one method embodiment for treating a bone fracture using an orthopedic bone tamp and bone stabilization material delivery device according to the present invention; and

FIG. 17 is one embodiment of an orthopedic bone tamp and bone stabilization material delivery kit according to the present invention.

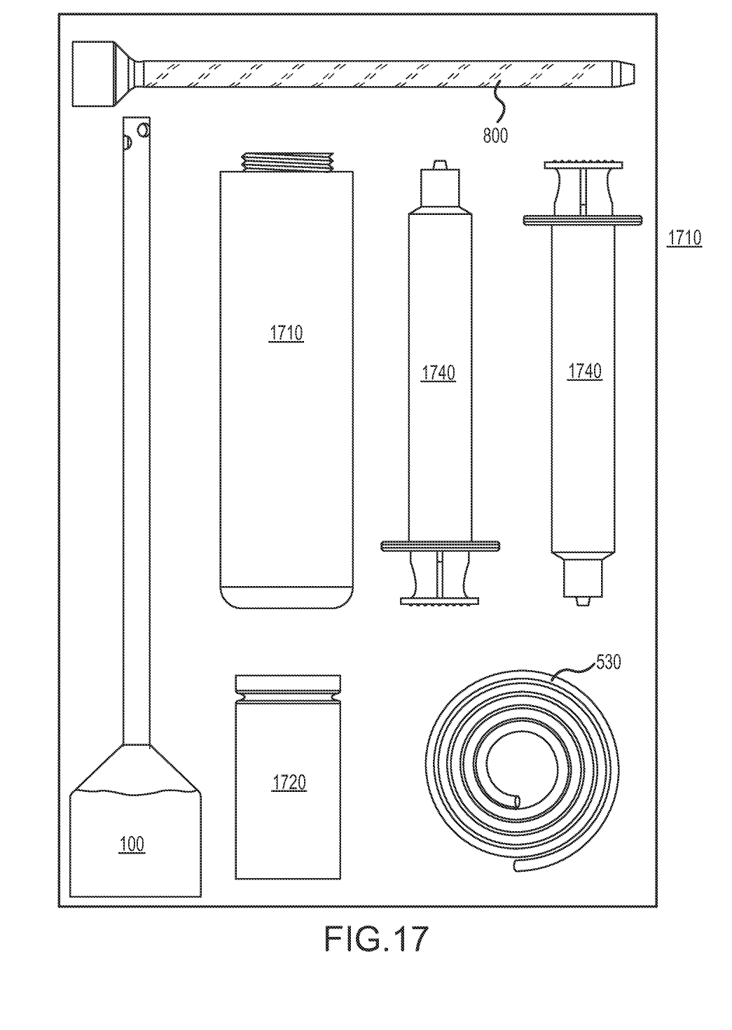

The Figures depict embodiments of the present invention for purposes of illustration only. One skilled in the art will readily recognize from the following discussion that alternative embodiments of the structures and methods illustrated herein may be employed without departing from the principles of the invention described herein.

### DESCRIPTION OF THE INVENTION

An orthopedic bone-reducing device (referred to herein as a tamp) and bone stabilization material delivery device and its associated use in treating bone fractures are disclosed hereafter by way of example. A bone tamp is a device used in orthopedic surgery to reduce fractures. A tamp is typically used inside the bone to elevate depressed areas after a fracture so as to assist fracture stabilization. Once the fractured components of a bone have been elevated to a natural or near natural position a stabilization material is inserted into the bone cavity. The bone tamp and stabilization material delivery device of the present invention is operable to concurrently elevate (reduce) fractured bone components while delivering bone stabilization material. As a result, the fractured bone components remain in their elevated, natural state until the bone can heal.

The innovations of the present invention are described herein with particular reference to an intra-articular depression fracture of the tibial plateau. As one of reasonable skill in the relevant art will appreciate, intra-articular depression factures can occur in locations other than the tibial plateau. An intra-articular depression fracture is one in which a fragment or group of bone fragments are pushed in to the interior of the bone at the joint. Joints such as the wrist, ankle and elbow can all experience intra-articular depression fractures. The largest joint in the human body is the knee. Accordingly, knee fractures, specifically tibial plateau fractures are often intra-articular depression fractures.

To better understand the various novel aspects of the present invention one must first consider the general morphology of a joint. Joints provide smooth, stable articulation between bones so that they may take on various tasks. Joints vary widely in their structure but share several common characteristics essential to their function. Joints comprise two end segments of bone bound together by a fibrous capsule. While the articulating surface of each bone at the joint is smooth the opposing surfaces may have variable areas of contact at different positions of joint motion. Joint stability relies on passive joint morphology and active stabilization. Disruption of any component of the joint can result in altered joint function. For example, displaced intra articular fractures are associated with gaps or steps in the joint surface. This variance in the morphology can affect stabilization, cause pain, and disrupt the effective motion of the joint.

The knee has several weight bearing surfaces. The primary loads in the knee pass from the femur to the tibia, with the curved surface of the femur resting on the relatively flat surface of the tibia. Like a mountain with a flat top, this flat surface is called the tibial plateau. This is a very sturdy surface, yet it is vulnerable to trauma and can break, normally as the result to a side blow to the knee. In such a case the femur acts as a hammer as it hits the plateau.

An intra articular tibial plateau fracture is a bone fracture or break in the continuity of the bone occurring in the proximal part of the tibia or shinbone called the tibial plateau. Such a fracture will likely affect the knee joint, stability and motion. The tibial plateau is a critical weight-bearing area located on the upper extremity of the tibia and is composed of two slightly concave condyles (medial condyle and lateral condyle) separated by an intercondylar eminence and the sloping areas in front and behind it. It can be divided into three areas: the medial tibial plateau (the part of the tibial plateau that is nearer to the center of the body and contains medial condyle), the lateral plateau (the part of the tibial plateau that is farthest away from the center of the body and contains the lateral condyle) and the central tibial plateau (located between the medial and lateral plateaus and contains intercondylar eminence). A standard tibial plateau fracture involves cortical interruption, depression or displacement of the articular surfaces of the proximal tibia without significant injury to the capsule or ligaments of the knee. As the compact bone associated with the plateau fractures it is depressed into the trabecular bone tissue. As an analogy one can imagine a hard shell around a Styrofoam interior. Upon fracture the bone fragments are imbedded and depressed into the interior trabecular bone tissue. These fractured components must be elevated back to their natural position to properly heal and reform the plateau. Depending on the degree of depression, the elevated fragments leave behind a void or space of crushed or depressed trabecular tissue. Filling this void concurrently while elevating the fragments is one object of the present invention.

Embodiments of the present invention are hereafter described in detail with reference to the accompanying Figures. In the Figures, like numbers refer to like elements throughout. Moreover, the sizes of certain lines, layers, components, elements or features may be exaggerated for clarity and are not necessary to scale or proportional. Reference to the applicable description may be necessary.

Although the invention is hereafter described and illustrated with a certain degree of particularity, it is understood that the present disclosure has been made only by way of example and that those skilled in the art can resort to numerous changes in the combination and arrangement of parts without departing from the spirit and scope of the invention. Also, descriptions of well-known functions and constructions are omitted for clarity and conciseness.

The terms and words used in the following description and claims are not limited to the bibliographical meanings, but are merely used by the inventor(s) to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents. To aid in clarity of understanding the meaning of the following terms are explained as applied to the present invention.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

The terminology used herein is for the purpose of describing particular embodiments only and is not intended to be limiting of the invention. As used herein, the singular forms “a,” “an” and “the” are intended to include the plural forms as well, unless the context clearly indicates otherwise. Thus, for example, reference to “a component surface” includes reference to one or more of such surfaces.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be also understood that when an element is referred to as being “on,” “attached” to, “connected” to, “coupled” with, “contacting”, “mounted” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper”, “inside”, “outside” and the like, may be used herein for ease of description to describe one element or feature's relationship to another element(s) or feature(s) as illustrated in the figures. It will be understood that the spatially relative terms are intended to encompass different orientations of a device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

An intra-articular fracture is a fracture that involves a joint surface. When a fracture involves the joint surface the fragments need to be perfectly reduced to avoid future arthritis of that joint.

Included in the description are flowcharts depicting examples of the methodology that may be used to treat fractured bones using the orthopedic bone tamp and bone stabilization material delivery device of the present invention. In the following description, it will be understood that one or more individuals can implement each block of the flowchart illustrations, and combinations of blocks in the flowchart illustrations, alone or in combination.

Blocks of the flowchart illustrations support combinations of means for performing the specified functions and combinations of steps for performing the specified functions. It will also be understood that each block of the flowchart illustrations, and combinations of blocks in the flowchart illustrations, can be implemented by special purpose hardware or systems that perform the specified functions or steps, or combinations of special purpose hardware and associated instructions.

Upon reading this disclosure, those of skill in the art will appreciate still additional alternative structural and functional designs for a system and process using an orthopedic bone tamp and bone stabilization material deliver device through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

It will also be understood by those familiar with the art, that the invention may be embodied in other specific forms without departing from the spirit or essential characteristics thereof. Likewise, the particular naming and division of the modules, managers, functions, systems, layers, features, attributes, methodologies, and other aspects are not mandatory or significant, and the mechanisms that implement the invention or its features may have different names, divisions, and/or formats.

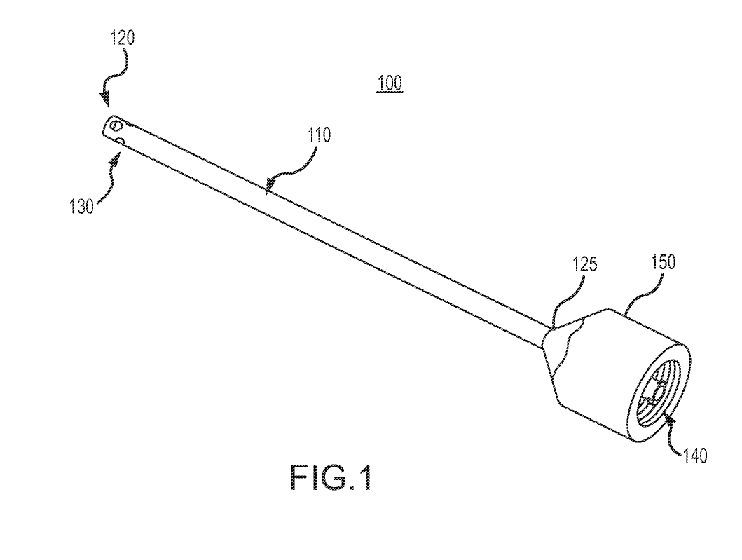

FIG. 1 presents a perspective view of an orthopedic bone tamp and bone stabilization material delivery device according to one embodiment of the present invention. The device 100 includes an extended cannula 110 connected at the proximal end 125 to a coupling device 150. The coupling device 150 includes, in this embodiment, a threaded means of receiving and securing additional equipment and a nozzle 140 that is fluidly coupled to the cannula 110. The cannula 110 includes at the distal end 120 a plurality of apertures or fenestrations 130 that traverse the walls of the cannula 110 so as to be fluidly connected to an interior lumen.

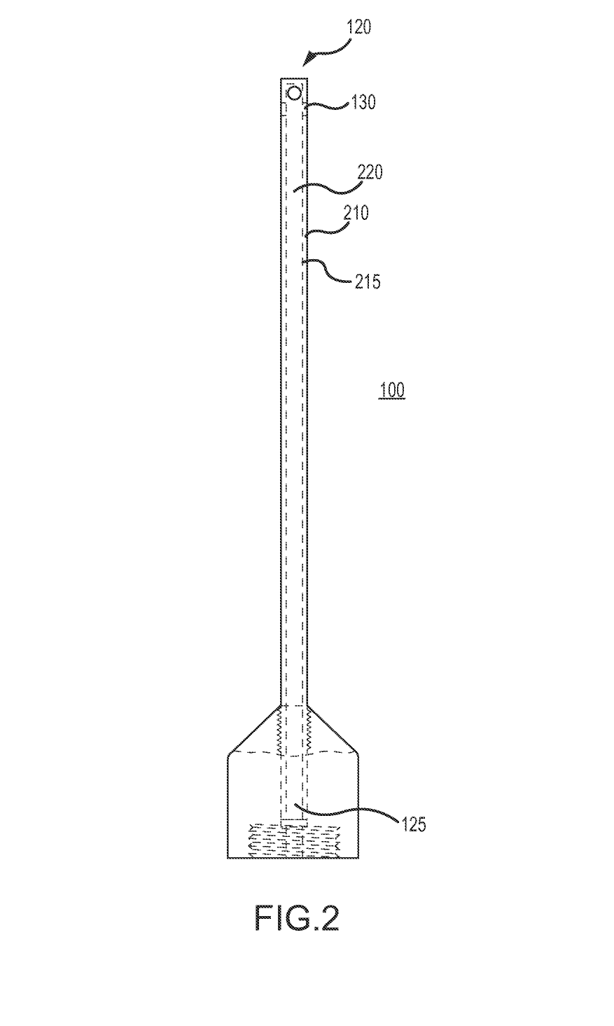

FIG. 2 presents a side cut away view for one embodiment of the orthopedic bone tamp and bone stabilization material delivery device according to the present invention. As shown the cannula 110 is comprised of an outer wall 210 and an inner wall 215 forming a lumen 220 that begins from the proximal end 125 of the cannula 110 terminating short of the distal end 120. The closed distal end 120 creates a bone displacement face that can manipulate bone fragments so as to reduce a fracture. Associated with the distal end 120 of the cannula 110 is a plurality of aperture or fenestrations 130. Each aperture 130 is fluidly coupled to the lumen 220 such that a bone stabilization material, traversing the lumen 220 from the proximal end 125 to the distal end 120 can be ejected from the cannula 110 in the immediate locality of the bone displacement face and the reduced bone fragment.

One skilled in the art will recognize that the cannula 110 and coupling device 150 can be constructed from a variety of material suitable for surgical applications. Materials include aluminum, stainless steel, titanium and titanium alloys, Chromium, and other metallic compounds that exhibit high strength and high rigidity as well as being biocompatible. Polymer based material demonstrating similar characteristics can also be used and are indeed contemplated by the present disclosure. As one of reasonable skill in the relevant art will recognize, the means by which the bone tamp enters and traverses a bone to gain access to the fractured region requires substantial strength and rigidity as well as the ability, when necessary, to transfer substantial force conveyed by the surgeon to the bone. The ability to identify and view the bone tamp as it traverses the body and bone is also a consideration for material selection. Fluoroscopy, ultrasound, endoscopy, tactile imaging and other means of medical imaging are used to assist the surgeon to position the bone tamp. Certain material can be more readily identified under certain types of imagery as opposed to other material. Consideration there is made to not only the ability to enter and manipulate the bone but the ability of the surgeon to observe the position of the device during the procedure.

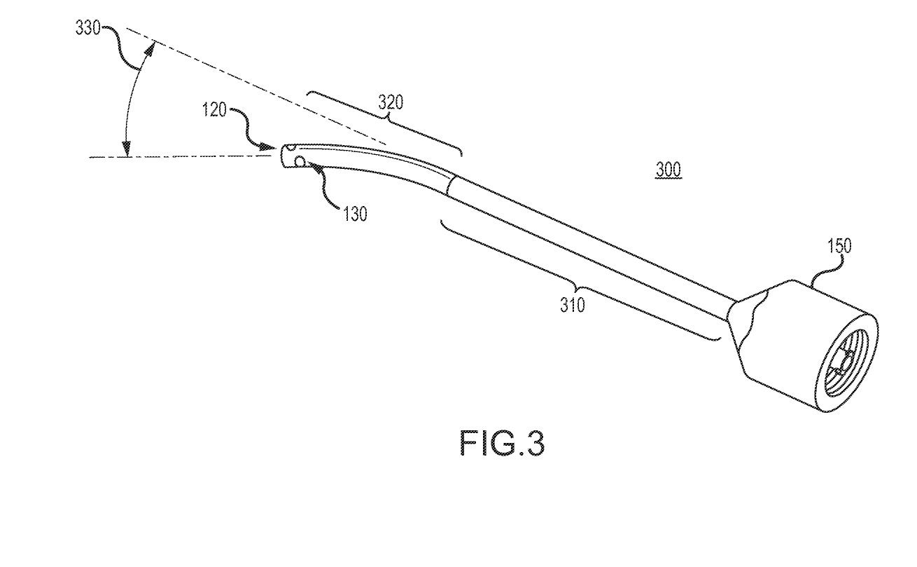

FIG. 3 presents another embodiment of a bone tamp and bone stabilization material delivery device of the present invention. The cannula 300 shown in FIG. 3 is comprised of a coupling device 150 as in the prior embodiment and a straight cannula portion 310. The straight portion 310 is connected or integrated with a curved or angular cannula portion 320. In the embodiment illustrated in FIG. 3, the curved portion 320 is angularly displaced 330 from the original longitudinal centerline of the cannula 310. One skilled in the art will appreciate that the angular displacement illustrated in Figure may vary according the nature of the fracture being treated.

In certain applications the reduction of fractured components cannot be effectively reached and manipulated by straight instruments. Once the compacted surface of the bone is traversed, the curved portion of the cannula 320 can be guided to otherwise inaccessible bone fragments resulting in more effective and complete reduction. As with the embodiment shown in FIG. 1, the curved portion of the cannula 320 includes a plurality of apertures 130 through which bone stabilization material can be introduced to the fracture zone concurrently with manipulation of the bone fragments. By doing so the fragments are reduced to their natural position and immediately stabilized.

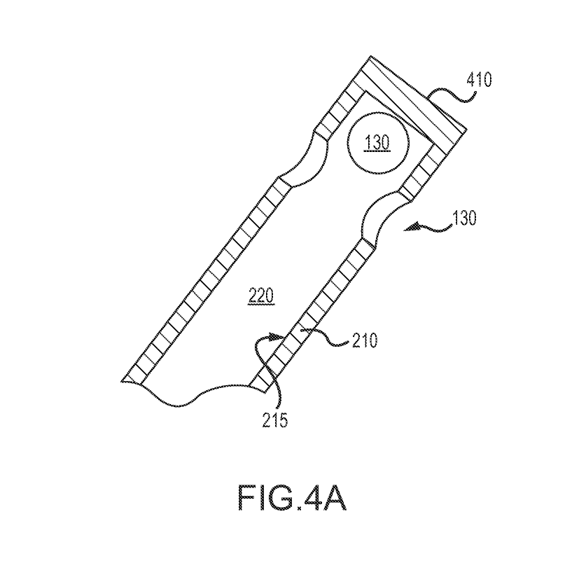

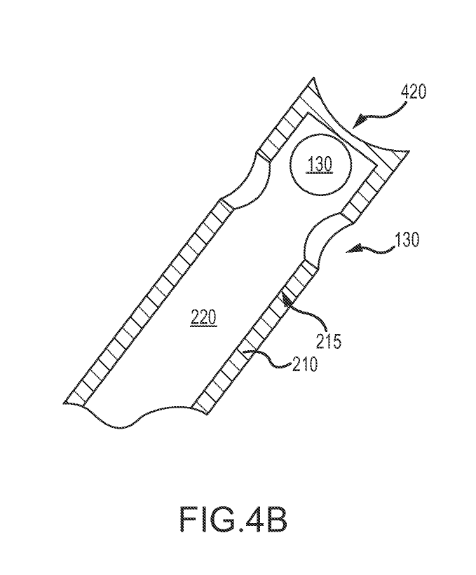

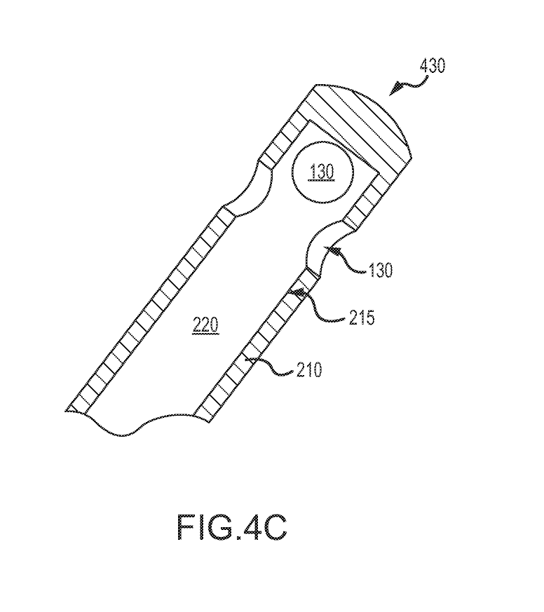

FIGS. 4A-4C show expanded views of various embodiments of the distal end of a bone tamp and bone stabilization material delivery device. As previously discussed the cannula 110 defines a lumen 220 beginning from the proximal end 125 and terminating short of the distal end 120. The closed face of the distal end 120 forms a bone displacement face operable to manipulate bone fragments.

FIG. 4A presents a first embodiment of a bone displacement face 410 located at the distal end 120 of the cannula 110. In the embodiment of the present invention shown in FIG. 4A the bone displacement face 410 is a planar surface substantially perpendicular to the longitudinal axis of the cannula 110. The circular planar face can used to manipulate bone pieces from their fractured location or a more natural orientation. Immediately below the planar face 410 are a plurality of apertures 130 and enable the bone stabilization material to exist the lumen 220 and stabilized the fracture zone.

FIG. 4B represents a second embodiment of the invention in which the bone displacement face 420 is concave in nature. The concave face curves inward toward the lumen 220 while maintaining a sealed or closed distal end 120. Again apertures 130 operable to deliver bone stabilization material are located immediately below the bone displacement face 420.

FIG. 4C shows a third bone displacement device face 430 having a convex shape. To manipulate bone fragments to their reduced position a surgeon uses the curved convex surface 430 of the cannula 110. As with the prior two embodiments the distal end 120 of the lumen 220 is closed, terminating with the bond displacement face 430. Apertures exist to introduce bone stabilization material immediately below the newly positioned bone fragment.

As introduced in herein, the cannula 110 defines a lumen 220 extending from the proximal end 125 to the apertures 130 associated with the distal end 120. The proximal end 125 includes a coupling device 150 configured to couple the cannula 110 to a plurality of attachments. Attachments can include a handle 510 or suitable apparatus to assist the surgeon in manipulating the bone tamp. The handle 510 can, in one embodiment of the present invention, present a surface by which to convey translational force to the bone tamp for insertion through the bone tissue so as to arrive at the fracture zone.

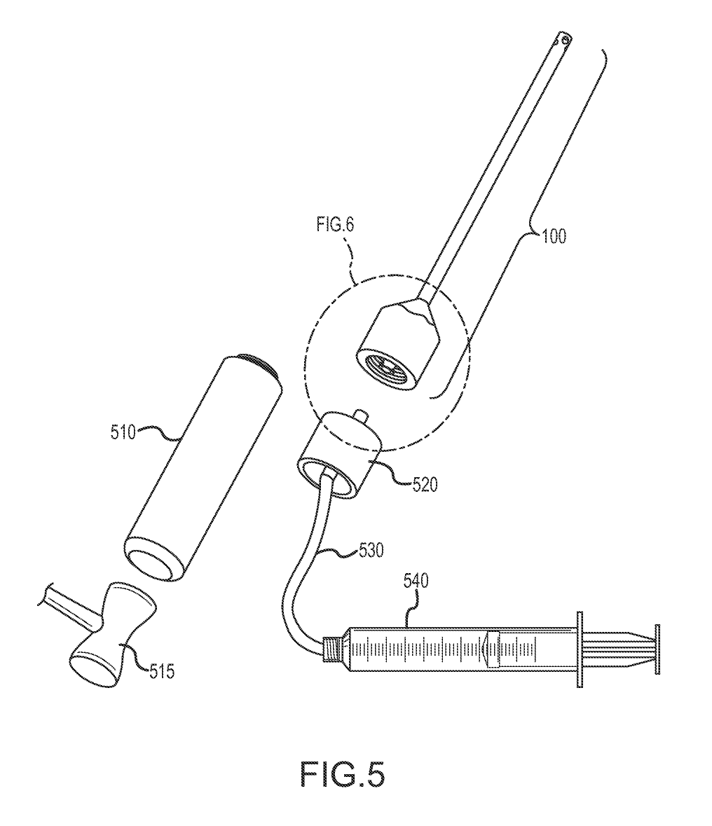

In another embodiment of the present invention a bone stabilization material link 520 can join the orthopedic bone tamp and bone stabilization device to a source 540 of bone stabilization material. For example and as shown in FIG. 5 the bone stabilization material link 520 can be couple to a hose or tube 530 which in thereafter coupled to a syringe 540 or similar device serving as a source of the bone stabilization material. As one of reasonable skill in the relevant art will appreciate the bone stabilization material link 520, tube 530 and syringe 540 are fluidly coupled to each other and upon insertion in to the coupling device 150 are coupled to the bone tamp 100.

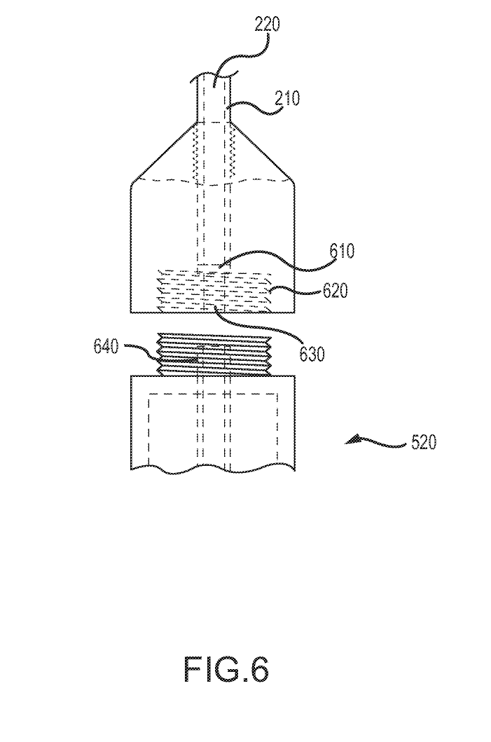

FIG. 6 shows an expanded view of the coupling device 150 of the orthopedic bone tamp 100, and the bone stabilization material link 520. In one embodiment of the present invention the lumen 220 of the bone tamp 100 includes at the proximal end a valve 610 and a receptacle or jack 630 that can be received by a corresponding plug 640 from the bone stabilization material link 520. The bone stabilization material link 520 couples with the bone tamp using, in one embodiment a threaded 620 connection to ensure that the jack 630 and plug 640 are firmly coupled. Once the connection is secure bone stabilization material can be conveyed from a source such as the depicted syringe 540 to the lumen 220 of the bone tamp 100.

Bone stabilization material is transported from its source to the fracture zone using the bone tamp of the present invention. In one embodiment of the present invention, bone fragments are manipulated from their displaced and/or depressed position to a natural, “pre-fracture” position. While holding the fragment in position bone stabilization material is transported from the source to the immediate vicinity of the fragment. Bone stabilization material fills the void resulting depression fracture stabilizing the newly positioned fragments. The present invention is operable to deliver a wide variety of bone stabilization material. As will be apparent to one of reasonable skill in the relevant art, material injected to the vicinity of a bone fracture serves to stabilize the region and to promote healing of the bone tissue. The stabilization or reinforcing material can be osteo-biological, osteo-conductive, osteo-inductive or osteo-genetic material, inorganic bone substitutes, synthetic polymers, or a matrix (combination) material. These materials can come in various forms, components and viscosities.

Inorganic bone substitutes can include calcium phosphate, tri-calcium phosphate, calcium sulfate, bioactive glasses and SiO2- and TiO2-based materials, coralline materials such as coralline hydroxyapatite, processed human bone, biphasic calcium phosphate, silicate bio-ceramic composite, and the like. Synthetic polymers include substances such as PMMA, polypeptide, polyglycolide, bioactive glasses, glass ionomers, sodium oxide, calcium oxide, phosphorous pentoxide and silicon dioxide, and the like. Osto-genetic material may include bone grafts, lab cultures, stem cells, osteoblasts, fibrin, etc.

In many cases bone cement is provided in two components. Dual component bone cements can include a powder format (i.e., pre-polymerized PMMA and or PMMA or MMA co-polymer beads and or amorphous powder, radio-opacifer, initiator) and a liquid (MMA monomer, stabilizer, inhibitor). The two components are mixed and a free radical polymerization occurs of the monomer when the initiator is mixed with the accelerator. The bone cement viscosity changes over time from a runny liquid into a dough-like state that can be safely applied and then finally hardens into solid hardened material. The set time can be tailored to help the physician safely apply the bone cement to treat osteoporotic compression fractures.

Alternatives to bone cement are also contemplated for use by the present invention. Biodegradable materials including protein-based materials (collagen, fibrin, thrombin, clotted blood), bone-graft, bone-graft substitutes and extenders (hydroxyapatite, beta-tricalcium phosphate, calcium sulfate, bioglass), and synthetic polymers (polyhanhydride, polylactide, polyglycolide, polyhydroxybutyrate-co-hydroxyvalerate, polyhydroxyalkanoate), as well as their combinations, can also be used to stabilize the fracture and delivered to the fracture zone via the orthopedic bone tamp and bone stabilization delivery device of the present invention.

The timely presentation and subsequent curing of the bone stabilization material is a significant component to the present invention. In the prior art once the fracture had been reduced the manipulative tool was removed often leaving a void or non-stable environment. The surgeon was faced with injecting into the area a stabilization product to secure the reduce fracture as soon as possible. But as implied above, the viscosity of various forms of stabilization material can vary widely. If the material possess a low viscosity even when properly and timely positioned within the fracture zone it will not stabilize the reduced fracture. And if the stabilization material has a high viscosity it may be difficult or impossible for the material to reach the proper location within the fracture zone before it becomes unworkable again rendering the fracture zone unstable.

As previously discussed one embodiment of the present invention is to provide a bone stabilization material delivery device that is physically integrated with a bone manipulative tamp so that immediately up reduction of a fracture, the area can be stabilized with the timely placement of stabilization material. Moreover the tamp or bone displacement face of the tamp can actively manipulate the fragment to maintain its proper location as the bone stabilization material cures.

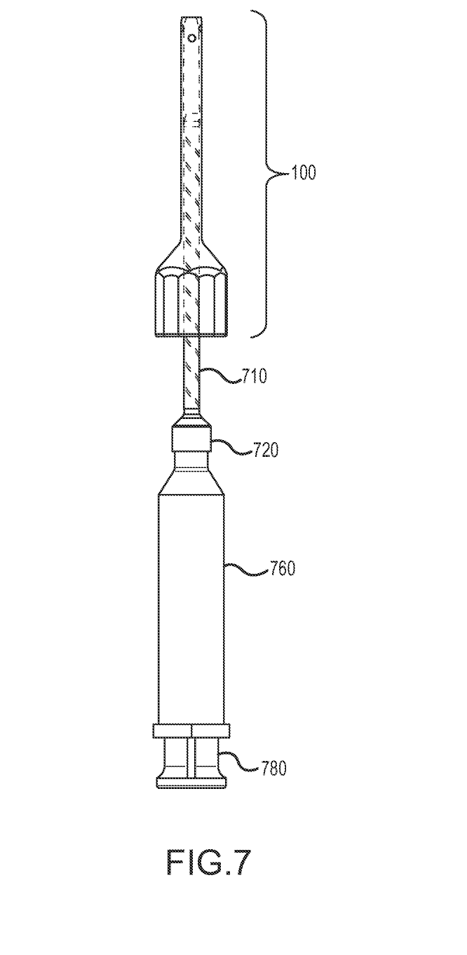

Premixed substances can be injected through the lumen of the orthopedic bone tamp using a syringe or similar device so as to be delivered to the fracture zone via the apertures 130 as the distal end 120 of the tamp 100. In another embodiment of the present invention, and as illustrated in FIG. 7, a delivery tube 710 is inserted within the cannula 110 that can convey the bone stabilization material to the apertures 130. The delivery tube 710 possesses an exterior diameter smaller than the interior diameter of the lumen 220. The delivery tube 710 includes a coupling fixture 720 that enables the delivery tube to be fluidly coupled to one or more bone stabilization material sources 760. In the example illustrated in FIG. 7 the delivery tube 710 is interposed within the lumen 220. The delivery tube 710 is coupled via its connector 720 to a syringe 760 containing bone stabilization materials. In the embodiment shown in FIG. 7 the syringe 760 includes two compartments that each house complementary components of the bone stabilization material. As the plunger 780 is depressed the substances are equally expelled into the delivery tube-coupling device 720. The coupling device, in this embodiment, is operable to mix the two components forming the bone stabilization material. The newly formed bone stabilization material is then conveyed to the apertures 130 of the cannula 110 via the delivery tube 710.

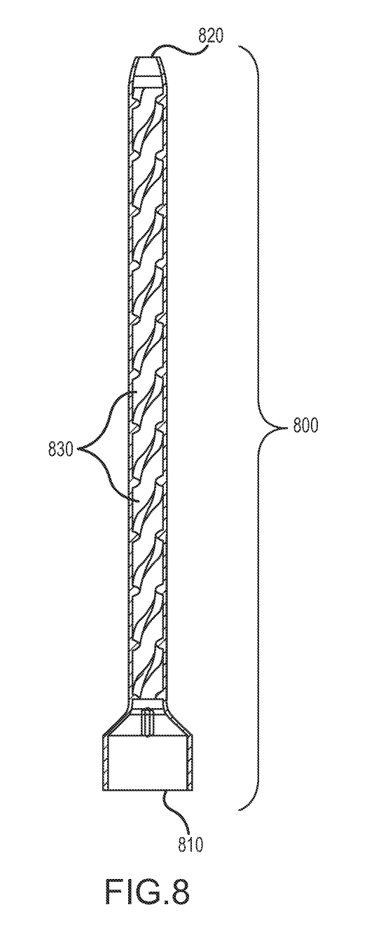

FIG. 8 is a side cut away view of one embodiment of a delivery device used for the delivery of bone stabilization material according to one embodiment of the present invention. The delivery device 800, comprising the delivery tube 710 and the delivery tube-coupling device 720, possesses a distal end 820 and a proximal end 810. In one embodiment of the present invention the interior of the delivery tube 710 includes aberrations 830 that promote mixing of the bone stabilization material as it traverses from the proximal end 810 to the distal end 820.

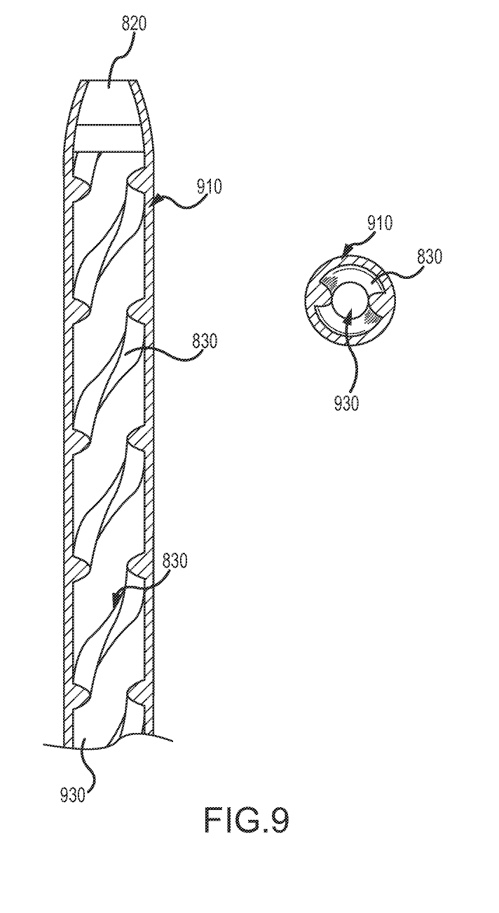

FIG. 9 represents an expanded cut away side and end view of the bone delivery tube, according to one embodiment of the present invention. The delivery tube 710 includes an outer wall 910 forming a delivery tube lumen 930. As previously mentioned the outer diameter of the outer wall 910 of the delivery tube 710 is less than the interior diameter of the cannula's 110 lumen 220. The delivery tube lumen 930 extends from, and throughout, the proximal end 810 to the distal end 820. Within the lumen 930 are included a series of aberrations 830 that produce a mixing or turbulent flow characteristic along throughout the lumen 930. In the embodiment shown in FIG. 9 the aberrations are configured in a spiraling format similar to rifling within the barrel of a gun. However in this case the aberrations 830 extend from the outer wall 910 into the lumen 930. FIG. 9 further presents an end view of the delivery tube 710 showing the outer wall 910, the aberrations 830 and the lumen 930 with an opening at the distal end 820.

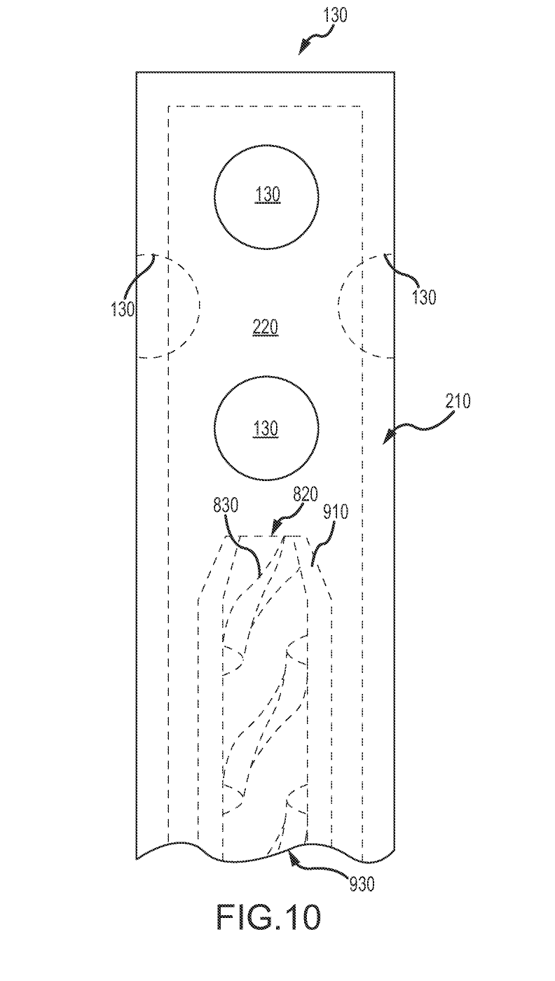

In a preferred embodiment the bone stabilization material delivery tube 710 extends within the lumen 220 of the cannula 110 such that the distal end 820 of the delivery tube 710 terminates proximate to the apertures 130. FIG. 10 presents, according to one embodiment of the present invention, an expanded side cut away view of a fully extended bone stabilization delivery device inserted within the lumen of an orthopedic bone tamp. As bone stabilization material travels down the delivery tube lumen 930, the bone stabilization material mixes and remains viable and employable. Upon exiting the delivery tube 710 the bone stabilization material enters the lumen 220 of the cannula 110 only to immediately be conveyed to the fracture zone via the apertures 130.

Should the bone stabilization material become overly viscous or unworkable, the delivery device 800 can be removed from the cannula 110 and replaced. This replacement can be accomplished while the bone displacement face 120 of the cannula 110 remains operative to position and maintain bone fragments in their natural position.

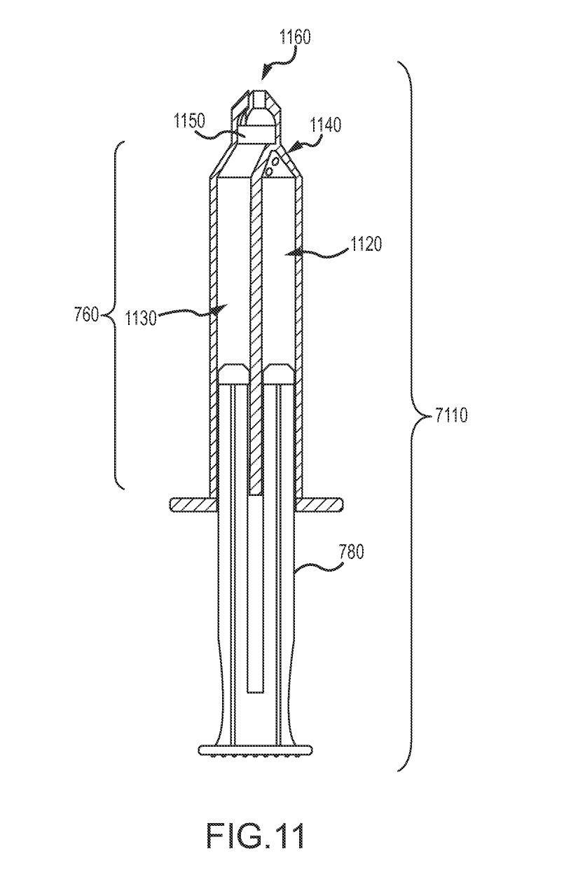

FIG. 11 shows one embodiment of a multi-compartment bone stabilization material syringe according to the present invention. As discussed many forms of bone stabilization material including bone cement are multi-component. Thus the bone stabilization material is inert until the two components are joined. Upon interaction of the components a chemical reaction forms the material into its final chemical compound and initiates a curing process. Ideally the material remains in a ductile and liquid state until delivered to the fracture zone where it cures and solidifies.

The syringe 1110 shown in FIG. 11 enables controlled delivery of, in this embodiment, two components. The body of the syringe 760 includes two compartments 1120, 1130 that house the bone stabilization material. As the plunger 780 is displaced into the compartments 1120, 1130, the bone stabilization material components are displaced from the syringe 160 and into a mixing chamber 1150. In one embodiment of the present invention one or more of the compartments 1120, 1130 includes a series of ports 1140 that are operable to control the mixture ratio and the rate of introduction. In some instances one component may be dry while the other is liquid. The ports 1140 can aid in providing the correct amount of liquid per dry component to achieve a proper blend. The syringe 110 of FIG. 11 further includes an angular exit port 1160 that presents the mixture to the lumen 930 of the delivery tube 710 with an angular momentum. As the mixture enters the lumen 930 it encounters the aberrations 830 which server to enhance the homogeneous nature of the mixture.

The treatment of a depression fracture such as a lateral tibial plateau facture is to reestablish joint stability, alignment, and articular congruity while preserving full range of motion. While each fracture is unique and there is no one universal treatment protocol it is well known within the medical community that for proper recovery the fixation of the tibial plateau must be rigid. Similarly other joint fractures of the classification also suggest a rigid reduction must be established to promote property healing

Should implants or placement of the fragments be loose or be provided inadequate fixation, intra-articular sepsis combined with fixation instability results in rapid chondrolysis and destruction of the joint. One skilled in art will recognize that while the present invention has been described with respect to a lateral tibial plateau fracture, the invention is equally applicable to other fracture treatment protocols. For example the bone tamp and bone stabilization delivery device of the present invention can be effectively employed to treat other articular fractures such as fractures of the wrist or distal radioulnar joint, the ankle including the talocrural joint, the subtalar joint and the inferior tibiofiblar joint, the hip or acetabulofemoral joint, and the shoulder or glenohumeral joint

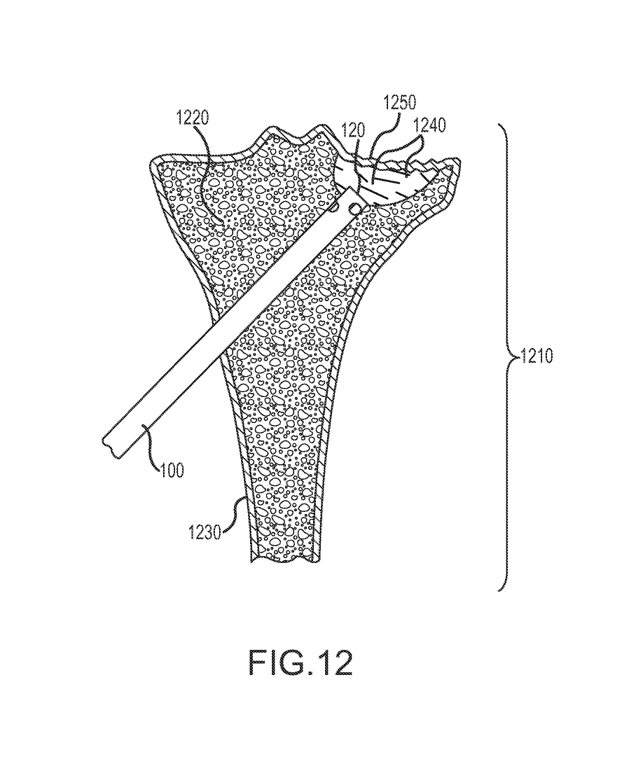

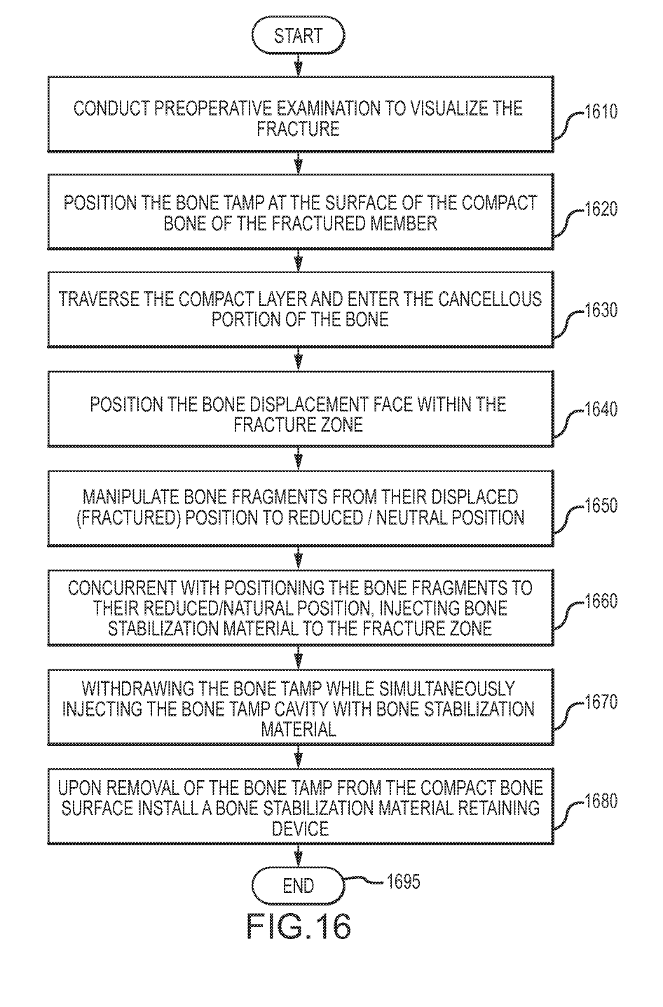

FIGS. 12-15 illustrate various stages of a method for using an orthopedic bone tamp and bone stabilization delivery device as applied to a lateral tibial plateau fracture. In combination with the flow chart of FIG. 16, these figures outline advantages and novel features of the present design.

FIG. 12 shows a side view of one embodiment of an orthopedic bone tamp and bone stabilization material delivery device in use to stabilize a lateral tibial plateau fracture according to the present invention. As shown the upper portion to the tibia 1210 includes a lateral tibial plateau 1250. The tibia, as will most bones, is comprised of a hard outer surface called the periosteum (not shown). This is a thin, dense outer membrane that contains nerves and blood vessels that nourish the bone. Second, is a layer called the compact bone 1230, which is smooth and hard and gives the bone is outer appearance. The inner bone, called cancellous layers 1220, is not as hard as the compact layer but is still very strong. And in certain bones an inner marrow layer exists for blood cell production.

The present invention uses an internal reduction technique by which fragments of the depressed bone 1240 are repositioned to their natural state 1250 using a bone displacement face 120. The bone tamp 100 enters the bone at a separate location by traversing the periosteum and compact bone and driving through the cancellous layer.

The bone tamp is advanced through the cancellous layer by applying axial force. Said differently, a handle 510, is secured to the proximal end 140 of the tamp 100 and a mallet or similar device is used to drive the displacement face/distal end 120 though but bone until it arrives at the fracture zone.

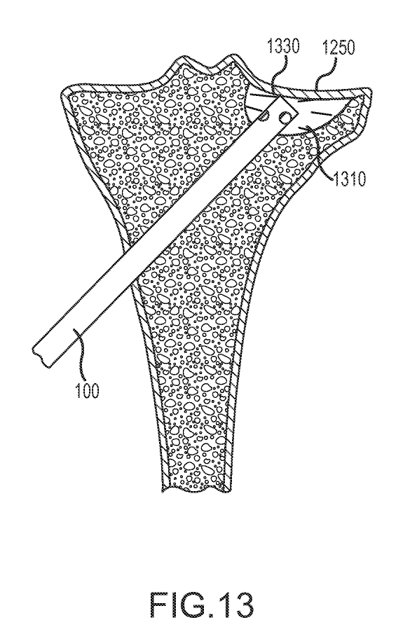

FIG. 13 shows an orthopedic bone tamp and bone stabilization material delivery device of the present invention traversing the tibia and placed in proximity to a plurality of bone fragments. In this rendering the bone fragments 1240 have been reduced to a new position 1330 that approximates that natural contour of the lateral tibial plateau. As one or reasonable skill in the relevant art will appreciate introduction and arrival of the bone displacement face to the fracture zone may take several hours of treatment. Indeed the placement of each fragment into its natural position may be an iterative process by which the bone displacement face is used to maneuver and reposition fragment orientations.

During this time the surgeon uses supplemental imagery from fluoroscopes or similar devices to gain a real time understanding of the position and movement of the bone tamp and bone displacement face within the bone. Accordingly material selection of the bone tamp of the present invention is chosen so as not only be visible under various forms of imagery but also for rigidity.

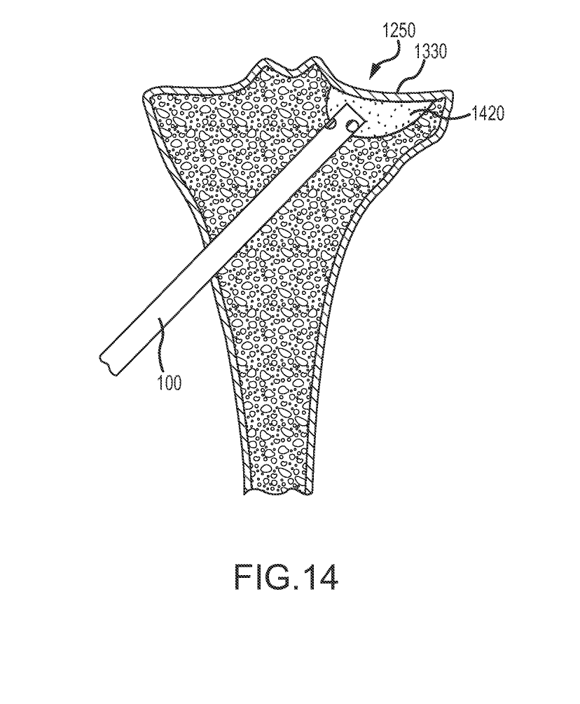

The treatment of a depressed lateral tibial plateau fracture continues by injecting within the facture zone a bone stabilization material. FIG. 14 shows a withdrawing orthopedic bone tamp and bone stabilization material delivery device of the present invention. In this depiction the bone fragments 1330 have been reduced to a quasi-natural or pre-fracture state 1250. Underneath the reduced new position of the bone fragments 1330 is the remnants of the fracture. In most instances the cancellous tissue has been crushed or damaged and can no longer support the compact bone fragments. According to one embodiment of the present invention bone stabilization material 1420 is introduced within the fracture zone as the bone tamp and bone displacement face withdraw.

With the bone fragments held in place by the bone displacement face, bone stabilization material 1420 is injected into the fracture zone immediately beneath and proximate to the fragments. As the bone tamp is removed the void left in the cancellous bone tissue is filled before the reduced bone fragments 1330 have an opportunity to migrate from their new position. Significantly the bone tamp is not removed nor is a new device introduced to supply bone stabilization material.

The process of withdraw/removal of the bone tamp continues as the bone tamp is withdrawn from the cancellous channel. Bone stabilization material is again introduced along with the retreating bone tamp so that the entirety of the cancellous tissue is stabilized.

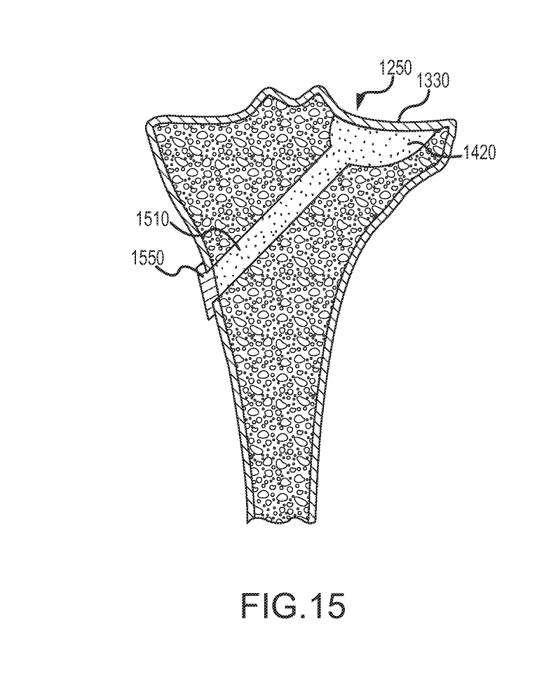

FIG. 15 presents a view of a lateral tibial plateau fracture reduced and stabilized by an orthopedic bone tamp and bone stabilization material delivery device of the present invention. As can be seen the bone fragments have been reduced 1330 to a near natural position 1250 and the fracture zone and bone tamp channel have been filled with bone stabilization material 1420, 1510. Upon exit from the bone tamp channel through the compact bone tissue, a bone stabilization material-retaining device 1550 is inserted into the cavity in the compact bone so as to keep the bone stabilization material within the confines of the bone. In one embodiment the bone stabilization retaining device is fitted on the exterior of the bone tamp and configured to slide down the length of the bone tamp such that immediately before the bone tamp exists the bone, the bone stabilization material retaining device 1550 enters the entry hole and seals it as the bone tamp is removed.

FIG. 16 presents one embodiment of a methodology for treatment of an articular fracture using an orthopedic bone tamp and bone stabilization material delivery device of the present invention. The process begins 1605 with a preoperative examination to gain visualization 1610 of the fracture. Based on this data and other examinations of the fracture a determination can be made as to the best course of treatment. In this example, an articular facture is detected and open reduction is elected as the best course of treatment.

Access to the fracture is accomplished via an incision in the skin, generally longitudinal and perpendicular to the axis of the joint. Access to a cortical window can also be established. Using fluoroscopy or other imaging techniques as would be known to one of reasonable skill in the relevant art, a strategy for elevating impacted articular fragments can be conceived.

In this example a decision is made to utilize the bone tamp of the present invention to manipulate and elevate (reduce) one or more impacted articular bone fragments. While the bone tamp could be introduced to the fracture zone through the facture an election is made to proceed via a cortical window. Accordingly the bone tamp is positioned on the surface of the compact bone proximal of the fracture 1620. Using a mallet or similar device by which to translate axial force to the bone tamp, the tamp traverses 1630 the compact bone layer and enters cancellous tissue on a trajectory to place the bone displacement face in a position to elevate the one or more articular fragments.

Using a mallet or similar tool, the bone tamp extends through 1640 the cancellous tissue of the bone into the fracture zone. Using imagery the surgeon manipulates the impacted and displaced articular fragments to their reduced/natural position 1650. As the articular fragments are elevated one of reasonable skill in the relevant art will appreciate that as a result of the fracture the stabilizing environment has also been damaged. The underlying cancellous tissue has likely been depressed or destroyed and even the presence of the bone tamp to some degree may undermine the natural stability characteristics of the underlying tissue. One advantage of the present invention is the introduction of bone stabilization material without removal of the bone tamp and reinsertion of a bone stabilization delivery device. As one of reasonable skill can appreciate the placement of bone stabilization material relative to the reduced fracture is critical, yet the process of removing the bone tamp and replacing it with a separate bone stabilization material delivery device is problematic. Despite fluoroscopy and similar imaging technology a certain degree of uncertainly exists as whether the bone stabilization material is being delivered to the proper local. Moreover there is a temporal lapse during which the reduced fragment may shift while waiting for stabilization. In the present invention the bone tamp remains proximate to the reduced fracture while the handle used to manipulate the bone tamp is removed and replaced with a bone stabilization material delivery system.

According to one embodiment of the present invention, bone stabilization material is concurrently injected 1660 into the fracture zone upon placement of the articulated fragment into its reduced position. The immediate introduction of stabilization material provides the newly elevated bone fragment with a stabile environment and minimizes additional displacement as additional support is added. In other embodiments of the present invention, conventional or locked plates, and/or lag screws, augment the introduction of bone stabilization material buttressing the fracture zone. These implementation methodologies are known within the art and the specifics of their application within the context of the present invention will be readily apparent to one of ordinary skill in the relevant art in light of this specification.

Having introduced bone stabilization material into the fracture zone with the articular bone fragments elevated and reduced, the treatment continues by withdrawing 1670 the bone tamp from the fracture zone while concurrently injecting additional bone stabilization material. In such a manner the damaged fracture zone and void created by the bone tamp itself are filled with bone stabilization material to promote recover of the fracture zone and maintain the integrity of the reduced fracture.

As the bone tamp reaches the compact bone interface a bone stabilization material retaining device is placed 1680 within the compact bone interface. The retaining device serves to secure the bone stabilization material within the interior of the bone. In one embodiment of the present invention, the retaining device is associated with the proximal end of the cannula of the bone tamp. As the cannula is extracted from the bone, the retainer slides down the circumference of the cannula so as to arrive at the distal end of the cannula as the distal end is extricated from the bone. As the cannula is extracted from the bone the retaining device is detached from the bone tamp/cannula filling the opening in the bone and securing the bone stabilization material within.

With the bone tamp removed and the retaining device in place, access to cortical window can be closed completing the use 1695 of the orthopedic bone tamp and bone stabilization material delivery device of the present invention. One of reasonable skill in the relevant art will recognize that additional procedures are associated with the reduction and treatment of an articulated fracture. While other support structures may be utilized to stabilize the fracture, the reduction of the fracture to create a stabilized a near normal plateau greatly enhances the potential recovery.

Disclosed herein is an orthopedic bone tamp and bone stabilization material delivery device suitable for use in the treatment of a depressed bone fracture. In many cases of bone trauma, the outer portion of the bone, commonly referred to as compact bone, is displaced or pushed into the underlying cancellous tissue. To achieve optimal recovery these displaced and depressed bone fragments must be elevated back to their normal position. Fracture reduction refers to the “re-alignment” of broken bone. Minimally invasive surgery manipulates the bone fragments to reconstruct the damage. In areas such as the joints the surface of the bone can collapse into the underlying tissue. It is also critical that this surface be carefully reconstructed to promote joint mobility and to reduce ongoing complications. However elevating such bone fragments into their correct position has long been a challenge. On significant impediment to achieving a stable reduction of such bone fragments is that upon proper placement of a depressed fragment by a bone tamp or an equivalent device, the fragment moves prior to the introduction of a stabilization material.

Embodiments of the present invention described herein presents an orthopedic bone tamp that is not only operable to elevate and reduce depressed bone fragment to a natural or near natural position, but also concurrently or substantially concurrently introduce bone stabilization material to the immediate vicinity of the newly reduced fragment. By doing so the reduce (repositioned) fragment remains in its proper location promoting a more efficient and likely more successful recovery.

Although the invention has been described and illustrated with a certain degree of particularity, it is understood that the present disclosure has been made only by way of example and that numerous changes in the combination and arrangement of parts can be resorted to by those skilled in the art without departing from the spirit and scope of the invention, as hereinafter claimed.

While there have been described above the principles of the present invention in conjunction with an orthopedic bone tamp and bone stabilization material delivery device, it is to be clearly understood that the foregoing description is made only by way of example and not as a limitation to the scope of the invention. Particularly, it is recognized that the teachings of the foregoing disclosure will suggest other modifications to those persons skilled in the relevant art. Such modifications may involve other features that are already known per se and which may be used instead of or in addition to features already described herein. Although claims have been formulated in this application to particular combinations of features, it should be understood that the scope of the disclosure herein also includes any novel feature or any novel combination of features disclosed either explicitly or implicitly or any generalization or modification thereof which would be apparent to persons skilled in the relevant art, whether or not such relates to the same invention as presently claimed in any claim and whether or not it mitigates any or all of the same technical problems as confronted by the present invention. The Applicant hereby reserves the right to formulate new claims to such features and/or combinations of such features during the prosecution of the present application or of any further application derived therefrom.

1. A bone tamp for use in surgical procedures, the tamp comprising:
a cannula having a tubular wall with an outside diameter and an inside diameter, a distal end, and a proximal end, wherein the distal end is closed and includes a bone displacement face with a bone displacement face diameter substantially equal to the outside diameter of the cannula;
a lumen defined by the tubular wall of the cannula traversing through the proximal end and terminating at the distal end at the bone displacement face; and
a plurality of apertures within the tubular wall proximate to the distal end and fluidly connected to the lumen.

2. The bone tamp of claim 1, wherein the bone displacement face is configured to transport bone from a fractured position to a natural position.

3. The bone tamp of claim 1, further comprising a coupling device proximate to the proximal end and adapted to introduce a bone stabilization material into the lumen.

4. The bone tamp of claim 3, wherein the bone stabilization material can be an osteo-biological material, an inorganic bone substitute, a synthetic material, or a matrix material chosen from a group consisting of pre-polymerized PMMA, MMA co-polymer beads, amorphous powder, radio-opacifer, liquid MMA monomer, collagen, fibrin, thrombin, clotted blood, bone-graft, bone-graft substitutes and extenders, hydroxyapatite, beta-tricalcium phosphate, tricalcium phosphate, bioactive glasses, glass ionomers, silicon oxide, calcium sulfate, bioglass, synthetic polymers, polyhanhydride, polylactide, polyglycolide, polyhydroxybutyrate-co-hydroxyvalerate and polyhydroxyalkanoate.

5. The bone tamp of claim 1, wherein the proximate end includes a coupling device adapted to fluidly couple the cannula to a source of a bone stabilization material.

6. The bone tamp of claim 5, wherein the source of the bone stabilization material comprises a mixing vessel having two or more chambers and wherein a first chamber includes a first part of a multipart bone stabilization material and a second chamber includes a second part of the multipart bone stabilization material and wherein the mixing vessel is operable to deliver to a proximal end of a delivery tube measured portions of the first part of the multipart bone stabilization material and the second part of the multipart bone stabilization material.

7. The bone tamp of claim 6, wherein the delivery tube includes a delivery tube tubular wall with an outside diameter and an inside diameter wherein the outside diameter of the delivery tube tubular wall is less that the inside diameter of the tubular wall of the lumen.

8. The bone tamp of claim 6, wherein the delivery tube is adapted to insert within the lumen of the cannula terminating short of the apertures.

9. The bone tamp of claim 6, wherein the delivery tube includes an internal helix and wherein responsive to the first part of the multipart bone stabilization material and the second part of the multipart bone stabilization material traversing the delivery tube from the proximal end to a distal end of the delivery tube, the helix is operable to mix the first part of the multipart bone stabilization material and the second part of the multipart bone stabilization material to a final bone stabilization material.

10. The bone tamp of claim 5, wherein the coupling device is configured to accept a handle suitable to manipulate the bone displacement face.

11. The bone tamp of claim 6, wherein the handle is adapted to transfer impact energy from the proximal end to the bone displacement face.

12. The bone tamp of claim 1, wherein the plurality of apertures are configured transversely across the tubular wall.

13. The bone tamp of claim 1, wherein the plurality of apertures are adapted to deliver a bone stabilization material.

14. The bone tamp of claim 1, wherein the plurality of apertures are operable to concurrently deliver a bone stabilization material as the bone displacement face positions bone from a fractured position to a natural position.

15. The bone tamp of claim 1, further comprising a bone sealing device sheathing the cannula at the proximate end and adapted to seal a void left by the removal of the cannula.

16. The bone tamp of claim 1, wherein the bone displacement face is substantially planar and perpendicular to the tubular wall.

17. The bone tamp of claim 1, wherein the bone displacement face is concave.

18. The bone tamp of claim 1, wherein the bone displacement face is convex.

19. A method for repositioning, in a fracture zone, fractured bone components to their natural position using a bone tamp, the method comprising;
establishing an opening in a bone using the bone tamp wherein the bone tamp is characterized as including a cannula with a tubular wall defining a lumen with an outside diameter and an inside diameter, a distal end, and a proximal end, and wherein the lumen terminates short of the distal end forming a bone displacement face and wherein the cannula includes a plurality of apertures within the tubular wall proximate to the distal end and fluidly connected to the lumen;
repositioning fractured bone components to substantially their natural position using the bone displacement face of the cannula;
coupling a source of bone stabilization material to the proximate end of the cannula;
delivering bone stabilization material through the lumen and the plurality of apertures to the fracture zone in support of the repositioned fractured bone components; and
removing the bone tamp from the opening in the bone.

20. The method for repositioning fractured bone using a bone tamp according to claim 19, wherein the proximate end of the cannula is associated with a coupling device configured to accept a handle suitable to manipulate the bone displacement face.

21. The method for repositioning fractured bone using a bone tamp according to claim 20, wherein the handle is adapted to transfer impact energy from the proximal end to the bone displacement face to establish the opening in the bone.

22. The method for repositioning fractured bone using a bone tamp according to claim 19, wherein delivering includes concurrently a bone stabilization material to the fracture zone as the bone displacement face positions fractured bone components to their natural position.

23. The method for repositioning fractured bone using a bone tamp according to claim 18, wherein delivering includes inserting into the lumen a delivery tube adapted to deliver bone stabilization material from the source to the apertures.

24. The method for repositioning fractured bone using a bone tamp according to claim 23 further comprising mixing within the delivery tube a first part of a multipart bone stabilization material and a second part of the multipart bone stabilization material.

25. The method for repositioning fractured bone using a bone tamp according to claim 19, wherein removing the bone tamp and delivering bone stabilization material occur concurrently.

26. The method for repositioning fractured bone using a bone tamp according to claim 19, wherein bone stabilization material includes an osteo-biological material, an inorganic bone substitute, a synthetic material, or a matrix material chosen from a group consisting of pre-polymerized PMMA, MMA co-polymer beads, amorphous powder, radio-opacifer, liquid MMA monomer, collagen, fibrin, thrombin, clotted blood, bone-graft, bone-graft substitutes and extenders, hydroxyapatite, beta-tricalcium phosphate, tricalcium phosphate, bioactive glasses, glass ionomers, silicon oxide, calcium sulfate, bioglass, synthetic polymers, polyhanhydride, polylactide, polyglycolide, polyhydroxybutyrate-co-hydroxyvalerate and polyhydroxyalkanoate.

27. The method for repositioning fractured bone using a bone tamp according to claim 19, further comprising inserting a bone seal into the opening in the bone upon removal of the bone tamp.

28. A bone reconstruction kit for the repositioning of fractured bone components in a fracture zone and concurrent introduction of bone stabilization material, the kit comprising:
a cannula having a tubular wall defining a lumen with an outside diameter and an inside diameter, a distal end, and a proximal end, wherein the lumen terminates short of the distal end forming a bone displacement face and wherein the cannula includes a plurality of apertures within the tubular wall proximate to the distal end and fluidly connected to the lumen;
a source of bone stabilization material and a bone stabilization material delivery system adapted to deliver the bone stabilization material from the source to the fracture zone via the lumen and the plurality of apertures;
a handle adapted to couple to the cannula via the coupling device and suitable to manipulate the bone displacement face; and
a coupling system proximate to the proximal end of the cannula; and operable to couple to the cannula a plurality of devices including the source of bone stabilization material and the handle.

29. The bone reconstruction kit according to claim 28, wherein the cannula is operable to concurrently introduce the bone stabilization material into a fracture zone as the bone displacement face positions one or more fractured bone components to their natural position.

30. The bone reconstruction kit according to claim 28, wherein the bone displacement face is configured to transport bone from a fractured position to a natural position.

31. The bone reconstruction kit according to claim 28, wherein bone stabilization material includes an osteo-biological material, an inorganic bone substitute, a synthetic material, or a matrix material chosen from a group consisting of pre-polymerized PMMA, MMA co-polymer beads, amorphous powder, radio-opacifer, liquid MMA monomer, collagen, fibrin, thrombin, clotted blood, bone-graft, bone-graft substitutes and extenders, hydroxyapatite, beta-tricalcium phosphate, tricalcium phosphate, bioactive glasses, glass ionomers, silicon oxide, calcium sulfate, bioglass, synthetic polymers, polyhanhydride, polylactide, polyglycolide, polyhydroxybutyrate-co-hydroxyvalerate and polyhydroxyalkanoate.

32. The bone reconstruction kit according to claim 28, wherein the coupling device is adapted to convey bone stabilization material from the source into the lumen.

33. The bone reconstruction kit according to claim 28, further comprising a bone seal operable to seal a bone opening upon removal of the bone tamp.

34. The bone reconstruction kit according to claim 28, wherein the bone displacement face is substantially planar and perpendicular to the tubular wall.

35. The bone reconstruction kit according to claim 28, wherein the bone displacement face is concave.

36. The bone reconstruction kit according to claim 28, wherein the bone displacement face is convex.

37. The bone reconstruction kit according to claim 28, wherein the source of bone stabilization material comprises a mixing vessel having two or more chambers and wherein a first chamber includes a first part of a multipart bone stabilization material and a second chamber includes a second part of the multipart bone stabilization material and wherein the mixing vessel is operable to deliver to a proximal end of a delivery tube measured portions of the first part of the multipart bone stabilization material and the second part of the multipart bone stabilization material.

38. The bone reconstruction kit according to claim 37, wherein the delivery tube includes a delivery tube tubular wall with an outside diameter and an inside diameter wherein the outside diameter of the delivery tube tubular wall is less that the inside diameter of the tubular wall of the lumen.

39. The bone reconstruction kit according to claim 37, wherein the delivery tube is adapted to insert within the lumen of the cannula terminating short of the apertures.

40. The bone reconstruction kit according to claim 37, wherein the delivery tube includes an internal helix and wherein responsive to the first part of the multipart bone stabilization material and the second part of the multipart bone stabilization material traversing the delivery tube from the proximal end to a distal end of the delivery tube, the helix is operable to mix the first part of the multipart bone stabilization material and the second part of the multipart bone stabilization material to a final bone stabilization material.
